(19) United States
(12) Patent Application Publication
(10) Pub. No.: US 2026/0234095 A1
(43) Pub. Date: Aug. 13, 2026

(54) PROCESSES FOR MAKING UNSATURATED ALIPHATIC HYDROCARBONS, INCLUDING VINYL ACETATE FROM ETHYLENE GLYCOL DIACETATE

(71) Applicant: New Product Innovation, LLC, Johnston, IA (US)

(72) Inventor: Ray Chrisman, Midland, MI (US)

(73) Assignee: New Product Innovation, LLC, Johnston, IA (US)

(21) Appl. No.: 19/537,181

(22) Filed: Feb. 11, 2026

Related U.S. Application Data
(60) Provisional application No. 63/757,016, filed on Feb. 11, 2025.
(60) Provisional application No. 63/757,045, filed on Feb. 11, 2025.

(51) Int. Cl. C07C 69/15; B01J 19/00; B01J 31/02; C08F 210/12

(52) U.S. Cl. CPC C07C 69/15; B01J 19/008; B01J 31/0237; C08F 210/12

(57) ABSTRACT

Disclosed herein are several processes for producing unsaturated aliphatic hydrocarbons from saturated aliphatic hydrocarbons using cavitation technology. Feedstock is passed through a cavitation apparatus to generate bubbles that collapse, creating localized high temperatures and pressures sufficient to crack the saturated hydrocarbon. The process may employ polar substituted feedstocks, non-polar or non-reactive additives with lower boiling points, or submicron particles to moderate energy release and improve selectivity. In one embodiment, ethylene glycol diacetate is converted to vinyl acetate monomer and acetic acid using hydrodynamic cavitation.

### CROSS-REFERENCES & RELATED APPLICATIONS

This application claims priority to U.S. Provisional Application No. 63/757,016, filed Feb. 11, 2025, and entitled “PROCESS FOR MAKING VINYL ACETATE FROM ETHYLENE GLYCOL DIACETATE,” and U.S. Provisional Application No. 63/757,045 filed Feb. 11, 2025, and entitled “PROCESSES FOR MAKING UNSATURATED ALIPHATIC HYDROCARBONS,” which is hereby incorporated by reference in their entirety under 35 U.S.C. § 119(e).

### BACKGROUND

Unsaturated aliphatic hydrocarbon-containing chemicals such as ethylene, propylene, butene, hexene, vinyl acetate, vinyl chloride, allyl acetate, and styrene are important intermediates for chemical processes to produce industrial and commercial chemicals and articles of manufacture. In general, these chemicals are made from unsaturated hydrocarbons by thermal cracking or catalytic cracking at high temperatures. The heat energy required for cracking represents a significant cost component affecting production economics. Interest therefore exists in producing such unsaturated aliphatic hydrocarbon-containing chemicals in an energy efficient manner. Interest also exists in using raw materials that can be derived from renewable resources to produce unsaturated aliphatic hydrocarbon-containing chemicals.

Cavitation such as ultrasonic cavitation and hydrodynamic cavitation have been proposed for numerous applications including for chemical processes such as for making biodiesel. Cavitation can, in some instances, generate radicals that can further react. Although produced radicals, e.g., from t-butanol, can combine to form a mixture of chemicals, and in some instances the mixture contains unsaturated hydrocarbons. The energy generated in cavitation has been used to cleave carbon-carbon bonds, e.g., for lowering the viscosity of residual oils and decomposition of organic pollutants. Cavitation has not proven to be sufficiently selective for converting saturated aliphatic hydrocarbons to unsaturated aliphatic hydrocarbons for commercial viability.

Vinyl acetate monomer (VAM) is an important commodity chemical. Numerous processes exist for making VAM. An interest exists in using sustainable feedstocks rather than fossil-based feedstocks to make commodity chemicals, including VAM. Commercial acceptance of commodity chemicals made using sustainable feedstocks will, in part, depend on the cost of their production.

Vinyl acetate is typically made by the catalytic reaction of ethylene, acetic acid, and oxygen. Some industrial production uses the hydroesterification of acetic acid and acetylene. A proposed route is the thermal or catalytic cracking of 1,1-diacetoxyethane. See, for instance, U.S. Pat. No. 2,425,389 and European Patent Application Publication No 0 048 173 A1. The high costs of producing 1,1-diacetoxyethane have not led to this proposed route achieving commercial viability for making VAM.

Providing an economically attractive route for making vinyl acetate monomer from ethylene glycol diacetate is desired as the ethylene glycol diacetate can be obtained from renewable as well as fossil-based feedstocks. Making vinyl acetate monomer from sustainable resources can proceed through several routes. For instance, ethylene glycol diacetate can be made by the esterification of monoethylene glycol. The conventional process for making monoethylene glycol is by cracking fossil-based feedstock to produce ethylene, partially oxidizing the ethylene-to-ethylene oxide and then hydrolyzing ethylene oxide to monoethylene glycol. The conventional process could use ethylene that is derived from sustainable feedstocks, e.g., ethylene made by the dehydration of ethanol derived from the fermentation of carbohydrate. Other processes exist for making monoethylene glycol from sustainable feedstocks.

The commercial viability of processes for making vinyl acetate monomer from ethylene glycol diacetate, whether from fossil sources or sustainable resources, will depend upon, among other things, the efficiencies of converting ethylene glycol diacetate to vinyl acetate monomer.

### BRIEF SUMMARY

This disclosure pertains to processes for making through cavitation, unsaturated aliphatic hydrocarbon-containing chemicals from saturated aliphatic hydrocarbon-containing chemicals. In various embodiments, cavitation processes are provided that enhance the selectivity of conversion of saturated aliphatic hydrocarbon-containing chemicals to unsaturated chemicals. Without intending to be limited by theory, it is believed that in a cavitation operation, bubbles are formed through the application of energy, e.g., sonic energy in the case of ultrasonic cavitation and kinetic energy in the case of hydrodynamic cavitation, and then the collapse of these bubbles, localized high temperatures and pressures are generated, say, in the range of 5000° K to 10,000° K and upwards of 50,000 kPa. It is believed that this localized energy generated in the cavitation operation can be moderated and focused on going from a single bond to a double bond between two carbon atoms and attenuate the generation of radicals or cleaving carbon-carbon bonds.

In accordance with various embodiments, this localized energy release is moderated to foster cracking. The moderation is effected by one or more of:
(i) providing a feedstock wherein the substituted, saturated aliphatic hydrocarbon-containing chemical is a polar chemical and wherein the feedstock has an essential absence of other polar components, although polar chemicals can be produced in the cavitation operation;
(ii) providing in the feedstock at least one non-polar chemical having a normal boiling point at least about 10° C., preferably at least about 20° C., lower than the normal boiling point of the substituted, saturated aliphatic hydrocarbon-containing chemical, and preferably the mass ratio of non-polar chemical to the substituted, saturated aliphatic hydrocarbon-containing chemical is in the range of about 0.5:10 to 10:1, say, about 1:10 to 1:1;
(iii) providing in the feedstock particles having a major dimension of less than 1 micron, preferably in the range of about 10 to 500 nanometers; and
(iv) providing a feedstock wherein the substituted, saturated aliphatic hydrocarbon-containing chemical is substituted on each carbon in a vicinal segment with electron withdrawing moieties.

In one broad aspect, which may forgo the use of an additive in a generally polar feed, the processes of disclosed herein for converting substituted, saturated aliphatic hydrocarbon-containing chemical in a feedstock to unsaturated aliphatic hydrocarbon-containing chemical, said substituted, saturated aliphatic hydrocarbon-containing chemical being a polar chemical and being represented by the structure:

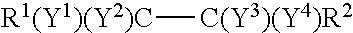

wherein R¹ and R² are the same or different and are selected from the group consisting of hydrogen and alkyl of one to six carbon atoms; Y¹, Y², Y³ and Y⁴ are the same or different and selected from the group consisting of hydrogen and an electron-withdrawing moiety selected from the group consisting of hydroxyl; halogen (especially F, Cl and Br); substituted and unsubstituted alkoxy of 1 to 4 carbons; substituted and unsubstituted acyl of 1 to 4 carbons; unsubstituted and substituted aryl of 6 to 20 carbons; sulfuryl; sulfonyl of 1 to 4 carbons; nitro of 0 to 4 carbons; and phosphoryl, and at least one of Y¹, Y², Y³ and Y⁴ is an electron withdrawing moiety, comprise:
passing the feedstock to a cavitation apparatus, said feedstock having an essential absence of polar compounds other than the substituted, saturated aliphatic hydrocarbon-containing chemical;
passing the feedstock through a cavitation apparatus to provide cavitation features and provide a two-phase liquid-gas mixture containing at least a major portion of the substituted, saturated aliphatic hydrocarbon-containing chemical in the liquid state and vapor phase bubbles then collapsing the bubbles resulting in localized areas of increased pressure and temperature sufficient to convert at least a portion of the saturated aliphatic hydrocarbon-containing chemical to unsaturated aliphatic hydrocarbon and provide a product mixture; and
separating unsaturated aliphatic hydrocarbon from the product mixture.

In some instances, the vapor phase bubbles contain a portion of the saturated aliphatic hydrocarbon-containing chemical. Typically, a carbon of the saturated aliphatic hydrocarbon-containing chemical that is substituted with at least one electron withdrawing moiety becomes double bonded with an adjacent carbon. In some instances, an electron withdrawing moiety is eliminated in making the unsaturated aliphatic hydrocarbon. In preferred embodiments, the saturated aliphatic hydrocarbon-containing chemical has a higher normal boiling point than that of the unsaturated aliphatic hydrocarbon product, and in some instances, the normal boiling point is at least 20° C. higher. Frequently the feedstock is at a temperature below the boiling point of the saturated aliphatic hydrocarbon-containing chemical at the pressure of the feedstock as it is passed to the cavitation apparatus, and in some instances at least 20° C. below the boiling point. In some instances, this temperature is above the normal boiling point of the unsaturated aliphatic hydrocarbon.

In another broad aspect, which may use a non-polar additive, the processes disclosed herein for converting substituted, saturated aliphatic hydrocarbon to unsaturated aliphatic hydrocarbon-containing chemical, said substituted, saturated aliphatic hydrocarbon-containing compound being represented by the structure:

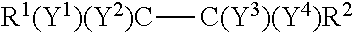

wherein R¹ and R² are the same or different and are selected from the group consisting of hydrogen and alkyl of one to six carbon atoms; Y¹, Y², Y³ and Y⁴ are the same or different and selected from the group consisting of hydrogen and an electron-withdrawing moiety selected from the group consisting of hydroxyl; halogen (especially F, Cl and Br); substituted and unsubstituted alkoxy of 1 to 4 carbons; substituted and unsubstituted acyl of 1 to 4 carbons; unsubstituted and substituted aryl of 6 to 20 carbons; sulfuryl; sulfonyl of 1 to 4 carbons; nitro of 0 to 4 carbons; and phosphoryl, and at least one of Y¹, Y², Y³ and Y⁴ is an electron withdrawing moiety, comprise:
admixing a saturated aliphatic hydrocarbon-containing feedstock with a substantially non-polar compound, which preferably has a normal boiling point less than that of saturated aliphatic hydrocarbon-containing compound, to provide a liquid-liquid mixture, said mixing being at a temperature and pressure sufficient to maintain both the feedstock and non-polar compound in the liquid phase;
passing the liquid-liquid mixture through a cavitation apparatus to provide cavitation features to provide a two-phase liquid-gas mixture containing the substituted, saturated aliphatic hydrocarbon-containing compound in the liquid state and the non-polar compound in the gaseous state in the form of vapor phase bubbles and then collapsing the bubbles resulting in localized areas of increased pressure and temperature sufficient to convert at least a portion of the saturated aliphatic hydrocarbon-containing compound to unsaturated aliphatic hydrocarbon and provide a product mixture; and
separating unsaturated aliphatic hydrocarbon from the product mixture.

The saturated aliphatic hydrocarbon-containing feedstock and substantially non-polar compound can be miscible, partially miscible or immiscible. Where partially miscible or immiscible, any non-dissolved, non-polar compound preferably is in the form of an emulsion in the feedstock with the feedstock comprising the continuous phase.

In yet another broad aspect, which may use a colloidal or particulate additive, the processes disclosed herein for converting substituted, saturated aliphatic hydrocarbon to unsaturated aliphatic hydrocarbon-containing chemical, said substituted, saturated aliphatic hydrocarbon-containing chemical being represented by the structure:

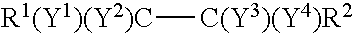

wherein R¹ and R² are the same or different and are selected from the group consisting of hydrogen and alkyl of one to six carbon atoms; Y¹, Y², Y³ and Y⁴ are the same or different and selected from the group consisting of hydrogen and an electron-withdrawing moiety selected from the group consisting of hydroxyl; halogen (especially F, Cl and Br); substituted and unsubstituted alkoxy of 1 to 4 carbons; substituted and unsubstituted acyl of 1 to 4 carbons; unsubstituted and substituted aryl of 6 to 20 carbons; sulfuryl; sulfonyl of 1 to 4 carbons; nitro of 0 to 4 carbons; and phosphoryl, and at least one of Y¹, Y², Y³ and Y⁴ is an electron withdrawing moiety, comprise:
admixing a saturated aliphatic hydrocarbon-containing chemical feedstock with particles having a major dimension of less than 1 micron, said admixing being sufficient to provide a substantially uniform colloidal suspension of said particles;
passing the colloidal suspension through a cavitation apparatus to provide cavitation features and provide a two-phase liquid-gas mixture containing at least a major portion of the substituted, saturated aliphatic hydrocarbon-containing chemical in the liquid state and vapor phase bubbles then collapsing the bubbles resulting in localized areas of increased pressure and temperature sufficient to convert at least a portion of the saturated aliphatic hydrocarbon-containing chemical to unsaturated aliphatic hydrocarbon and provide a product mixture; and
separating unsaturated aliphatic hydrocarbon from the product mixture.

The particles can provide nucleation sites for the formation of bubbles and can foster the collapse of the bubbles. The particles can be substantially inert or catalytic. Whether or not catalytic, the particles enhance at least one of the rate of conversion of the saturated aliphatic hydrocarbon and the selectivity of the conversion to unsaturated aliphatic hydrocarbon. Often the particles are provided in a mass ratio to the feedstock of about 0.001:1 to 0.05:1.

In another broad aspect, which may use vicinal substitution, the processes disclosed herein for converting substituted, saturated aliphatic hydrocarbon to unsaturated aliphatic hydrocarbon-containing chemical, said substituted, saturated aliphatic hydrocarbon-containing compound being represented by the structure

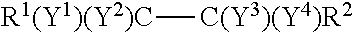

wherein R¹ and R² are the same or different and are selected from the group consisting of hydrogen and alkyl of one to six carbon atoms; Y¹, Y², Y³ and Y⁴ are the same or different and selected from the group consisting of hydrogen and an electron-withdrawing moiety selected from the group consisting of hydroxyl; halogen (especially F, Cl and Br); substituted and unsubstituted alkoxy of 1 to 4 carbons; substituted and unsubstituted acyl of 1 to 4 carbons; unsubstituted and substituted aryl of 6 to 20 carbons; sulfuryl; sulfonyl of 1 to 4 carbons; nitro of 0 to 4 carbons; and phosphoryl, and preferably, at least one of Y¹ and Y² and at least one of Y³ and Y⁴ is an electron withdrawing moiety, comprise:
passing the feedstock to a cavitation apparatus, said feedstock comprising the substituted, saturated aliphatic hydrocarbon-containing chemical;
passing the feedstock through a cavitation apparatus to provide cavitation features and provide a two-phase liquid-gas mixture containing at least a major portion of the substituted, saturated aliphatic hydrocarbon-containing chemical in the liquid state and vapor phase bubbles then collapsing the bubbles resulting in localized areas of increased pressure and temperature sufficient to convert at least a portion of the saturated aliphatic hydrocarbon-containing chemical to unsaturated aliphatic hydrocarbon and provide a product mixture; and separating unsaturated aliphatic hydrocarbon from the product mixture.

In some instances, at least one of R¹ and R² is hydrogen, i.e., the saturated aliphatic hydrocarbon would provide unsaturated aliphatic hydrocarbon where the unsaturation is at the alpha and beta carbons.

The cavitation apparatus can be any suitable apparatus for generating cavitation conditions such as apparatus using ultrasound or microwave energy and hydrodynamic cavitation apparatus can be stationary or rotational such as orifice, vortex and rotational apparatus. Usually, hydrodynamic cavitation apparatus are used due to lower energy costs to generate similar cavitation. In each of these apparatus, operating variables exist to optimize the processes for conversion to unsaturated aliphatic hydrocarbon. For instance, with ultrasonic cavitation apparatus, the power and frequency can be varied to change the density and size of bubbles being generated. Typically, the lowering of the frequency of the ultrasound reduces the density of the bubbles but the bubbles are larger than those generated at a higher frequency. The energy released by the collapse of the bubbles increases with increased bubble size. Hence, frequency can be optimized to facilitate achieving high selectivities to the unsaturated aliphatic compound.

Hydrodynamic cavitation is influenced by parameters of temperature, pressure, flow rate, viscosity, and geometry as is well known by those skilled in cavitation technology.

Example 1 relates to a process for converting ethylene glycol diacetate to vinyl acetate comprising admixing an ethylene glycol diacetate-containing feedstock with a substantially non-reactive compound having a normal boiling point less than that of ethylene glycol diacetate to provide a liquid-liquid mixture; passing the liquid-liquid mixture through a hydrodynamic cavitation apparatus to provide cavitation features to provide a two-phase liquid-gas mixture containing the ethylene glycol diacetate in the liquid state and the non-reactive compound in the gaseous state in the form of vapor phase bubbles by reducing the pressure to at or below the liquid/vapor pressure threshold for the non-reactive compound then collapsing the bubbles resulting in localized areas of increased pressure and temperature sufficient to convert at least a portion of the ethylene glycol diacetate to vinyl acetate and acetic acid and provide a product mixture; and separating vinyl acetate from the product mixture.

Example 2 relates to Examples 3-15, wherein the cavitation apparatus is a stationary or rotational cavitation apparatus.

Example 3 relates to Examples 1-2 and 4-15, wherein the cavitation apparatus is an orifice or vortex cavitation apparatus.

Example 4 relates to Examples 1-3 and 5-15, wherein the non-reactive compound is acetic acid.

Example 5 relates to Examples 1~4 and 6-15, wherein the non-reactive compound comprises a hydrocarbon having from 5 to 16 carbons and a normal boiling point between about 80° and 220° C.

Example 6 relates to Examples 1-5 and 7-15, the non-reactive compound is present in an amount of about 0.5 to 10 mass percent of the ethylene glycol diacetate-containing feedstock.

Example 7 relates to Examples 1-6 and 8-15, the temperature of the liquid-liquid mixture as it is passed to the cavitation apparatus is between about 50° and 150° C.

Example 8 relates to Examples 1-7 and 9-15, wherein the pressure of the liquid-liquid mixture as it is passed to the cavitation apparatus is between about 200 to 50,000 kPa absolute.

Example 9 relates to Examples 1-8 and 10-15, wherein the liquid-liquid mixture contains a nucleating agent.

Example 10 relates to Examples 1-9 and 11-15, wherein the nucleating agent is an inert gas which is dissolved in the liquid.

Example 11 relates to Examples 1-10 and 12-15, wherein the cavitation apparatus has two or more cavitation zones in series.

Example 12 relates to Examples 1-11 and 13-15, wherein a portion of the ethylene glycol diacetate is converted and the product mixture comprises unreacted ethylene glycol diacetate, acetic acid, vinyl acetate and non-reactive compound, further comprising separating vinyl acetate and acetic acid from the product mixture by distillation and providing a bottoms fraction comprising ethylene glycol diacetate, and passing at least a portion of the bottoms fraction to the cavitation apparatus.

Example 13 relates to Examples 1-12 and 14-15, wherein the non-reactive compound comprises acetic acid.

Example 14 relates to Examples 1-13 and 15, wherein the bottoms fraction contains acetic acid.

Example 15 relates to Examples 1-14, wherein the non-reactive compound comprises a hydrocarbon having from 5 to 16 carbons and a normal boiling point between about 80° and 220° C.

Example 16 relates to a process for converting substituted, saturated aliphatic hydrocarbon-containing chemical in a feedstock to unsaturated aliphatic hydrocarbon-containing chemical, said substituted, saturated aliphatic hydrocarbon-containing chemical being represented by the structure: R¹(Y¹)(Y²)C—C(Y³)(Y⁴)R² wherein R¹ and R² are the same or different and are selected from the group consisting of hydrogen and alkyl of one to six carbon atoms; Y¹, Y², Y³ and Y⁴ are the same or different and selected from the group consisting of hydrogen and an electron-withdrawing moiety selected from the group consisting of hydroxyl; halogen; substituted and unsubstituted alkoxy of 1 to 4 carbons; substituted and unsubstituted acyl of 1 to 4 carbons; unsubstituted and substituted aryl of 6 to 20 carbons; sulfuryl; sulfonyl of 1 to 4 carbons; nitro of 0 to 4 carbons; and phosphoryl, and at least one of Y¹, Y², Y³ and Y⁴ is an electron-withdrawing moiety, comprising passing the feedstock to a cavitation apparatus, said feedstock having an essential absence of polar compounds other than the substituted, saturated aliphatic hydrocarbon-containing chemical; passing the feedstock through a cavitation apparatus to provide cavitation features and provide a two-phase liquid-gas mixture containing at least a major portion of the substituted, saturated aliphatic hydrocarbon-containing chemical in the liquid state and vapor phase bubbles then collapsing the bubbles resulting in localized areas of increased pressure and temperature sufficient to convert at least a portion of the saturated aliphatic hydrocarbon-containing chemical to unsaturated aliphatic hydrocarbon and provide a product mixture; and separating unsaturated aliphatic hydrocarbon from the product mixture.

Example 17 relates to Examples 16 and 18-21, wherein the substituted, saturated aliphatic hydrocarbon-containing chemical is a polar chemical.

Example 18 relates to Examples 16-17 and 19-21, further comprising admixing a saturated aliphatic hydrocarbon-containing feedstock with a substantially non-polar compound to provide a liquid-liquid mixture, said mixing being at a temperature and pressure sufficient to maintain both the feedstock and non-polar compound in the liquid phase.

Example 19 relates to Examples 16-18 and 20-21, further comprising admixing a saturated aliphatic hydrocarbon-containing chemical feedstock with particles having a major dimension of less than 1 micron, said admixing being sufficient to provide a substantially uniform colloidal suspension of said particles.

Example 20 relates to Examples 16-19 and 21, wherein the cavitation apparatus is a stationary or rotational hydrodynamic cavitation apparatus.

Example 21 relates to Examples 16-20, wherein the halogen is chosen from the group consisting of fluorine, chlorine, and bromine.

While multiple embodiments are disclosed, still other embodiments of the disclosure will become apparent to those skilled in the art from the following detailed description, which shows and describes illustrative embodiments of the disclosed system, devices, and methods. As will be realized, the disclosed system, devices, and methods are capable of modifications in various obvious aspects, all without departing from the spirit and scope of the disclosure. Accordingly, the drawings and detailed description are to be regarded as illustrative in nature and not restrictive.

### DETAILED DESCRIPTION

All patents, published patent applications, and articles referenced herein are hereby incorporated by reference in their entirety.

### Definitions

As used herein, the following terms have the meanings set forth below unless otherwise stated or clear from the context of their use.

Where ranges are used herein, the end points only of the ranges are stated so as to avoid having to set out at length and describe each and every value included in the range. Any appropriate intermediate value and range between the recited endpoints can be selected. By way of example, if a range of between 0.1 and 1.0 is recited, all intermediate values (e.g., 0.2, 0.3, 0.63, 0.815 and so forth) are included as are all intermediate ranges (e.g., 0.2-0.5, 0.54-0.913, and so forth).

The use of the terms “a” and “an” is intended to include one or more of the elements described.

Admixing or admixed means the formation of a physical combination of two or more elements which may have a uniform or non-uniform composition throughout and includes, but is not limited to, solid mixtures, solutions and suspensions.

Aliphatic hydrocarbon means organic compounds in which carbon atoms are connected by single, double or triple bonds to form non-aromatic structures. As used herein, aliphatic compounds include compounds containing aromatic substituents, and thus an alkaryl compound is an aliphatic compound.

Cavitation features means providing a two-phase liquid-gas mixture containing vapor phase bubbles and then collapsing the bubbles resulting in localized areas of increased pressure and temperature. In specific embodiments, it may mean providing a two-phase liquid-gas mixture containing the ethylene glycol diacetate in the liquid state and the non-reactive compound in the gaseous state in the form of vapor phase bubbles by reducing the pressure to at or below the liquid/vapor pressure threshold for the non-reactive compound then collapsing the bubbles resulting in localized areas of increased pressure and temperature.

Cracking means the formation of a double bond as opposed to splitting a compound between carbon atoms.

Ethylene glycol diacetate is also known as 1,2-diacetoxyethane and is abbreviated herein as GDA.

The half acetate ester of ethylene glycol is also known as 2-hydroxyethyl acetate and is abbreviated herein as HEA.

Sustainable resources are plants and animals, including, but not limited to, waste products from plants and animals, and sustainable feedstocks are derived from sustainable resources.

Sustainable resources also include carbon dioxide used as a feedstock in processes to make monoethylene glycol, whether that carbon dioxide is captured from direct air capture or from emissions from facilities that emit carbon dioxide, including, but not limited to, incineration, power generation, fermentation, chemical and other industrial processes.

Cavitation reactors are well known. See, for instance, Zheng, et al., Recent Developments in Hydrodynamic Cavitation Reactors: Cavitation Mechanism, Reactor Design and Applications, Engineering, 2022, Volume 19, Issue 12, and Cako, et al, Cavitation Based Cleaner Technologies for Biodiesel and Processing of Hydrocarbon Streams: A Perspective on Key Fundamentals, Missing Process Data and Economic Feasibility—A Review, Ultrasonics Sonochemistry, 88 (2022)106081. In cavitation, the generation, growth and collapse of bubbles in liquids results in energy release in a localized area. In hydrodynamic cavitation, a liquid stream is passed into a lower pressure area, which pressure is at or below that at which the lower boiling component boils. Bubbles are generated and, once the liquid passes out of the lower pressure area, which is sudden, the bubbles collapse releasing energy.

Examples of hydrodynamic cavitation reactors are stationary reactors and rotational reactors. Stationary reactors include orifice and venturi reactors where liquid is pumped through a small cross-sectional area whereby linear flow rate is increased and into an expansion region having a larger cross-sectional area. Vortex cavitation reactors typically introduce the liquid tangentially and the pressure lowers at the center of the vessel. In rotational cavitation reactors a solid object is rotated within a liquid. While ultrasonic induced cavitation (acoustic cavitation) is capable of generating cavitation conditions, scalability for commercial application has been problematic.

The Unsaturated Aliphatic Hydrocarbon May have the Formula:

$${R}^{1} [ ( {Y}^{1} ) or ( {Y}^{2} ) ] C = C [ ( {Y}^{3} ) or ( {Y}^{4} ) ] {R}^{2}$$

wherein R¹, R², Y¹, Y², Y³ and Y⁴ are as defined above. Preferably where at least two of Y¹, Y², Y³ and Y⁴ of the saturated aliphatic hydrocarbon-containing compound are electron withdrawing groups, at least one of the electron withdrawing groups is retained in the unsaturated aliphatic hydrocarbon. Table I provides examples of substituted, aliphatic hydrocarbons and unsaturated aliphatic hydrocarbons generated by cracking.

TABLE I

| | |
|---|---|
| Saturated | Unsaturated |
| aliphatic hydrocarbon | aliphatic hydrocarbon |

| | |
|---|---|
| Chloroethanol | Vinyl chloride |
| 1,2-diacetoxypropane | Allyl acetate |
| 1-chloro-2-hydroxypropane | Allyl Chloride |
| Ethylene dichloride | Vinyl chloride |
| 1,1-diacetoxyethane | Vinyl acetate |
| 1,2-diacetoxyethane | Vinyl acetate |
| Hydroxyethylacetate | Vinyl acetate |
| Ethylbenzene | Styrene |
| 1,2-Diacetoxybutane | Buteneacetates |
| Ethanol | Ethylene |
| Propanol | Propylene |
| n-Butanol | Butene |
| n-Hexanol | Hexene |

Various embodiments may use various selectivity mechanisms to guide the cracking reaction to preferentially react at specific chemical sites. These selectivity mechanisms may include using a polar hydrocarbon-containing chemical in the feedstock, adding a non-polar additive to the feedstock, adding a colloidal or particulate additive to the feedstock, using a hydrocarbon-containing feedstock that has chemicals with electron-withdrawing moieties on carbons in a vicinal segment.

In embodiments where saturated aliphatic hydrocarbon-containing feedstock is admixed with a substantially nonpolar compound, the non-polar compound preferably has a lower boiling point and serves to generate the bubbles in the cavitation reactor. The non-polar compound is often an organic compound having a normal boiling point in between about 25° C. or 80° C. and 220° C. In any event, the non-polar compound should be in the liquid phase as it is supplied to the cavitation apparatus and is withdrawn from the cavitation apparatus. Examples of non-polar compounds include, but are not limited to, hydrocarbons having from about 5 to 16 carbon atoms, especially paraffins such as n-pentane, n-octane, isooctane, n-decane, n-pentene, and cetane, and aromatics such as benzene and the like. The amount of non-polar compound used will depend upon the boiling point of the non-polar compound with greater amounts being used where the boiling point of the non-polar compound is higher. In general, between about 0.5 and 10, say, 0.75 to 5, parts by mass are used per 100 parts by mass of substituted, saturated aliphatic hydrocarbon-containing feedstock.

A nucleating agent may be provided in the feedstock. The nucleating agent serves to provide sites for the bubbles of the non-reactive organic compound to form and grow. Nucleating agents can be solid particles or non-condensable, inert gas bubbles. Examples of inert gases include, but are not limited to, methane, nitrogen, and argon. In most instances gaseous nucleating agent is present in an amount of less than 100, say, 20, parts per million by mass based on the mass of the substituted, saturated aliphatic hydrocarbon-containing compound. Often, the gaseous nucleating agent is dissolved in the liquid phase and may be at saturation or oversaturation concentrations but still dissolved.

As discussed above, particles can be used as nucleation sites for the generation and for the collapse of bubbles. In some instances, the particles can also serve to moderate the high, localized temperatures generated by the cavitation to provide improved conditions for promoting cracking to the unsaturated aliphatic hydrocarbon product. In some instances, the particles can sorb the substituted, saturated aliphatic hydrocarbon-containing compound and thus provide it at localized high temperature conditions for the cracking. If desired the particles can be catalytically active for cracking to an unsaturated compound. Examples of particles include, but are not limited to, silica, carbon, metals and metal oxides such as aluminum, alumina, zirconia, magnesia, platinum, palladium, iron, iron oxide, copper, copper oxide, nickel, cobalt, chromium oxide, gallium oxide, gold, titanium, titania, tungsten, vanadium, molybdenum, lanthanum and mixtures thereof. Catalytically active moieties can be supported, say, on silica, alumina, silica-alumina such as zeolites), zirconia or carbon particles. Examples of catalytically active particles include, but are not limited to, gallium oxide on alumina, chromium oxide on silica, iron on ZSM-5, gold on silica, platinum and tin on magnesia, platinum indium on silica, nickel on alumina, nickel on silica, nickel on activated carbon, and nickel gallium on alumina.

The conditions for the cavitation reaction will vary depending upon the type of reactor and its design and upon the desired degree of conversion of the saturated aliphatic hydrocarbon-containing compound as those skilled in the art of cavitation reactors well understand. The optimization of conditions can readily be achieved by the artisan having the benefit of the instant disclosure.

For rotational cavitation apparatus, the design and speed of the rotating device are primary variables as the cavitation depends upon the generation of a low-pressure region on the trailing edge of the rotating device, e.g., an impeller, where the bubbles are formed and then promptly collapse. For stationary cavitation reactors, the cavitation feature is provided by a quick change in speed of the liquid causing a surrounding low pressure region enabling bubbles to form which are then collapsed as the liquid flow normalizes to a new steady state velocity. Regardless of whether the apparatus is a rotational or stationary device, the formation and then collapse of the bubbles is extremely fast, often less than 1 second. The localized pressures and temperatures resulting from the collapse, which conditions may last for milliseconds, are sufficient to thermally crack the saturated aliphatic hydrocarbon to an unsaturated aliphatic hydrocarbon. The cavitation apparatus may be run continuously or intermittently, for instance in a pulsed fashion.

With rotational cavitation apparatus, the surrounding pressure may be ambient pressure or below or above atmospheric pressure as the difference in liquid flow velocity and pressure differential is created by the rotating device. Pressure is a primary variable affecting the flow speed of the liquid through the orifice or vortex, and it is the differential in liquid flow speeds that achieves the cavitation features.

The temperature of the liquid to be subjected to cavitation features can vary over a wide range, and is generally at least ambient temperature, and is frequently between about 50° C. to 150° C., say, about 70° C. to 120° C. The factors influencing the temperature include the temperature of the feedstock, heat generated by pumping or moving the liquid, and the viscosity of the feedstock. Temperature can influence the cavitation features, with higher temperatures, e.g., above 150° C. being less preferred. Viscosity of the liquid can be adjusted by increasing or decreasing the temperature, giving the artisan another variable to optimize the cavitation features for the conversion to the unsaturated aliphatic hydrocarbon. Typically, the viscosity is in the range of 0.1 to 50, say 0.2 to 30, centipoise.

In some instances, the Cavitation Number (dimensionless) is in the range of about 0.1 to 1, even in the range of about 0.1 to 0.8. See Zheng, et al., supra, for a description of this parameter which takes into account the recovered static pressure from the device, vapor pressure of the non-reactive liquid, pressure, density of the feedstock and flow velocity of the liquid at the constriction. In general, more cavitation is achieved with higher flow velocities at the constriction provided by the orifice or vortex or rotational speed.

It is to be understood that two or more cavitation devices may be used in flow direction series, and these devices may be the same or different, e.g., an orifice device followed by a venturi device. Ultrasonic cavitation can be used simultaneously with hydrodynamic cavitation, and techniques such as microwave radiation and thermal heating can be used in conjunction with cavitation to provide additional energy.

Generally, only a portion of the substituted, saturated aliphatic hydrocarbon-containing compound is converted, e.g., less than about 50, and sometimes between about 5 and 30 percent. The unsaturated aliphatic hydrocarbon can be separated by any suitable manner including sorption and distillation. Unreacted, substituted aliphatic hydrocarbon-containing compound can be recycled to the cavitation apparatus.

In some embodiments, the ethylene glycol diacetate-containing feedstock may contain other components such as propylene diacetate and 1,2-butane diacetate. The ethylene glycol diacetate often is between about 50 and essentially 100 mass percent of the total diacetate diesters of vicinal glycols of 2 to 4 carbons in the feedstock. Generally, the feedstock is relatively free of water, e.g., less than about 500, and preferably less than about 100, parts per million by mass of total diacetate esters of vicinal glycols of 2 to 4 carbons. Acetic half esters of vicinal glycols of 2 to 4 carbons may also be present in the feedstock. In some instances, they constitute less than 1 mass percent of the feedstock.

The ethylene glycol diacetate-containing feedstock is admixed with a substantially non-reactive compound that has a lower boiling point and serves to generate the bubbles in the cavitation reactor. The non-reactive compound can be inorganic or organic. In most instances, the non-reactive compound is an organic compound having a normal boiling point in between about 80° and 220° C. Examples of non-reactive organic compounds include, but are not limited to, acetic acid and hydrocarbons having from about 5 to 16 carbon atoms such as n-pentane, n-octane, isooctane, n-decane, n-pentene, cetane, benzene, toluene, naphthalene, and the like. Sulfur dioxide is an example of a substantially non-reactive inorganic compound. The amount of non-reactive compound used will depend upon the boiling point of the non-reactive compound with greater amounts being used where the boiling point of the non-reactive compound is higher. In general, between about 0.5 and 10, say, 0.75 to 5, parts by mass are used per 100 parts by mass of ethylene glycol diacetate.

A nucleating agent may be provided in the ethylene glycol diacetate-containing feedstock. The nucleating agent serves to provide sites for the bubbles of the non-reactive organic compound to form and grow. Nucleating agents can be solid, inert particles or, preferably, non-condensable, inert gas bubbles. Examples of inert gases include, but are not limited to, methane, nitrogen, and argon. In most instances the nucleating agent is present in an amount of less than 100, say, 20, parts per million by mass based on the mass of the ethylene glycol diacetate-containing feed. Often, the gaseous nucleating agent is dissolved in the liquid phase and may be at saturation or oversaturation concentrations but still dissolved.

The conditions for the hydrodynamic cavitation reaction will vary depending upon the type of reactor and its design and upon the desired degree of conversion of the ethylene glycol diacetate as those skilled in the art of cavitation reactors well understand. The optimization of conditions can readily be achieved by the artisan having the benefit of the instant disclosure.

For rotational cavitation apparatus, the design and speed of the rotating device are primary variables as the cavitation depends upon the generation of a low-pressure region on the trailing edge of the rotating device, e.g., an impeller, where the bubbles are formed and then promptly collapse. For stationary cavitation reactors, the cavitation feature is provided by a quick change in speed of the liquid causing a surrounding low pressure region enabling bubbles to form which are then collapsed as the liquid flow normalizes to a new steady state velocity. Regardless of whether the apparatus is a rotational or stationary device, the formation and then collapse of the bubbles is extremely fast, often less than 1 second. The localized pressures and temperatures resulting from the collapse, which conditions my last for milliseconds, are sufficient to thermally crack ethylene glycol diacetate to vinyl acetate monomer.

In some embodiments, the cavitation apparatus may be run continuously or intermittently, for instance in a pulsed fashion.

With rotational cavitation apparatus, the surrounding pressure may be ambient pressure or below or above atmospheric pressure as the difference in liquid flow velocity and pressure differential is created by the rotating device. For stationary cavitation apparatus, the pressure of the liquid fed to the apparatus is frequently between about 200 to 50,000, say, between about 500 and 10,000, kPa absolute. The pressure is a primary variable affecting the flow speed of the liquid through the orifice or vortex, and it is the differential in liquid flow speeds that achieves the cavitation features.

The temperature of the liquid to be subjected to cavitation features can vary over a wide range, and is generally at least ambient temperature, and is frequently between about 50° C. to 150° C., say, about 70° C. to 120° C. The factors influencing the temperature include the temperature of the ethylene glycol diacetate-containing feedstock, heat generated by pumping or moving the liquid, and the viscosity of the feedstock. Temperature can influence the cavitation features, with higher temperatures, e.g., above 150° C. being less preferred. Viscosity of the liquid can be adjusted by increasing or decreasing the temperature, giving the artisan another variable to optimize the cavitation features for the conversion of ethylene glycol diacetate to vinyl acetate monomer. Typically, the viscosity is in the range of 0.1 to 50, say 0.2 to 30, centipoise.

In some instances, the Cavitation Number (dimensionless) is in the range of about 0.1 to 1, even in the range of about 0.1 to 0.8. See Zheng, et al., supra, for a description of this parameter which takes into account the recovered static pressure from the device, vapor pressure of the non-reactive liquid, pressure, density of the feedstock and flow velocity of the liquid at the constriction. In general, more cavitation is achieved with higher flow velocities at the constriction provided by the orifice or vortex or rotational speed.

It is to be understood that two or more cavitation devices may be used in flow direction series, and these devices may be the same or different, e.g., an orifice device followed by a venturi device.

The cracking provides vinyl acetate monomer and acetic acid. If propylene glycol diacetate and/or 1,2-diacetoxybutane are present, the corresponding unsaturated esters, allyl acetate and acetoxybutane such as one or more of 1-acetoxy-2-butene, 2-acetoxy-1-butene, and 2-acetoxy-2-butene.

Generally, only a portion of the ethylene glycol diacetate is converted, e.g., less than about 50, and sometimes between about 5 and 30, percent. Vinyl acetate monomer having a normal boiling point of 72° C. can readily be separated from the unreacted ethylene glycol diacetate by distillation. The distillation may be flash distillation or fractional distillation. The distillation can also serve to remove essentially all, or a portion of, the generated acetic acid. Where acetic acid is used to form bubbles, its presence in the unreacted ethylene glycol diacetate can be useful if the unreacted ethylene glycol diacetate is recycled to the cavitation apparatus. If another substantially non-reactive compound is used to form bubbles, it also is preferably retained in the bottoms fraction, i.e., the unreacted ethylene glycol diacetate fraction.

In some embodiments, the bottoms fraction is recycled to the cavitation apparatus.

The vinyl acetate monomer can be recovered from the overhead of the distillation by any suitable means. For instance, the vapor phase comprising vinyl acetate, acetic acid and potentially other components is withdrawn from the distillation and subjected to further distillation to provide at least an acetic acid rich side stream and a vinyl acetate rich stream. Preferably the vinyl acetate rich stream contains less than 0.1, most preferably less than 0.01, mass percent allyl acetate and less than 50 parts per million by mass of any compound containing four or more carbons bonded in series.

Although the disclosure has been described with reference to preferred embodiments, persons skilled in the art will recognize that changes may be made in form and detail without departing from the spirit and scope of the disclosed apparatus, systems and methods.

What is claimed is:

1. A process for converting substituted, saturated aliphatic hydrocarbon-containing chemical in a feedstock to unsaturated aliphatic hydrocarbon-containing chemical, said substituted, saturated aliphatic hydrocarbon-containing chemical being represented by the structure: 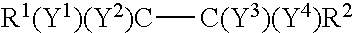
wherein R¹ and R² are the same or different and are selected from the group consisting of hydrogen and alkyl of one to six carbon atoms; Y¹, Y², Y³ and Y⁴ are the same or different and selected from the group consisting of hydrogen and an electron-withdrawing moiety selected from the group consisting of hydroxyl; halogen; substituted and unsubstituted alkoxy of 1 to 4 carbons;
substituted and unsubstituted acyl of 1 to 4 carbons; unsubstituted and substituted aryl of 6 to 20 carbons; sulfuryl; sulfonyl of 1 to 4 carbons; nitro of 0 to 4 carbons; and phosphoryl, and at least one of Y¹, Y², Y³ and Y⁴ is an electron-withdrawing moiety, comprising:
passing the feedstock to a cavitation apparatus, said feedstock having an essential absence of polar compounds other than the substituted, saturated aliphatic hydrocarbon-containing chemical;
passing the feedstock through a cavitation apparatus to provide cavitation features and provide a two-phase liquid-gas mixture containing at least a major portion of the substituted, saturated aliphatic hydrocarbon-containing chemical in the liquid state and vapor phase bubbles then collapsing the bubbles resulting in localized areas of increased pressure and temperature sufficient to convert at least a portion of the saturated aliphatic hydrocarbon-containing chemical to unsaturated aliphatic hydrocarbon and provide a product mixture; and
separating unsaturated aliphatic hydrocarbon from the product mixture.

2. The process of claim 1, wherein the substituted, saturated aliphatic hydrocarbon-containing chemical is a polar chemical.

3. The process of claim 1, further comprising admixing a saturated aliphatic hydrocarbon-containing feedstock with a substantially non-polar compound to provide a liquid-liquid mixture, said mixing being at a temperature and pressure sufficient to maintain both the feedstock and non-polar compound in the liquid phase.

4. The process of claim 1, further comprising admixing a saturated aliphatic hydrocarbon-containing chemical feedstock with particles having a major dimension of less than 1 micron, said admixing being sufficient to provide a substantially uniform colloidal suspension of said particles.

5. The process of claim 1 wherein the cavitation apparatus is a stationary or rotational hydrodynamic cavitation apparatus.

6. The process of claim 1, wherein the halogen is chosen from the group consisting of fluorine, chlorine, and bromine.

7. A process for converting substituted, saturated aliphatic hydrocarbon to unsaturated aliphatic hydrocarbon-containing chemical, said substituted, saturated aliphatic hydrocarbon-containing compound being represented by the structure 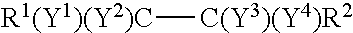
wherein R¹ and R² are the same or different and are selected from the group consisting of hydrogen and alkyl of one to six carbon atoms; Y¹, Y², Y³ and Y⁴ are the same or different and selected from the group consisting of hydrogen and an electron-withdrawing moiety selected from the group consisting of hydroxyl; halogen; substituted and unsubstituted alkoxy of 1 to 4 carbons; substituted and unsubstituted acyl of 1 to 4 carbons; unsubstituted and substituted aryl of 6 to 20 carbons; sulfuryl; sulfonyl of 1 to 4 carbons; nitro of 0 to 4 carbons; and phosphoryl, and preferably, at least one of Y¹ and Y² and at least one of Y³ and Y⁴ is an electron withdrawing moiety, comprising:
passing the feedstock to a cavitation apparatus, said feedstock comprising the substituted, saturated aliphatic hydrocarbon-containing chemical;
passing the feedstock through a cavitation apparatus to provide cavitation features and provide a two-phase liquid-gas mixture containing at least a major portion of the substituted, saturated aliphatic hydrocarbon-containing chemical in the liquid state and vapor phase bubbles then collapsing the bubbles resulting in localized areas of increased pressure and temperature sufficient to convert at least a portion of the saturated aliphatic hydrocarbon-containing chemical to unsaturated aliphatic hydrocarbon and provide a product mixture; and
separating unsaturated aliphatic hydrocarbon from the product mixture.

8. The process of claim 7 wherein at least one of R¹ and R² is hydrogen.

9. The process of claim 7 wherein the cavitation apparatus has two or more cavitation zones in series.

10. The process of claim 7 wherein the unsaturated aliphatic hydrocarbon product is represented by:
$${R}^{1} [ ( {Y}^{1} ) or ( {Y}^{2} ) ] C = C [ ( {Y}^{3} ) or ( {Y}^{4} ) ] {R}^{2}$$
wherein at least one of the electron withdrawing groups is retained in the unsaturated aliphatic hydrocarbon.

11. The process of claim 10 wherein the saturated aliphatic hydrocarbon-containing compound is 1,2-diacetoxyethane and the unsaturated aliphatic hydrocarbon product is vinyl acetate.

12. The process of claim 10 wherein the saturated aliphatic hydrocarbon-containing compound is 2-chloroethanol and the unsaturated aliphatic hydrocarbon product is vinyl chloride.

13. The process of claim 7, wherein the halogen is chosen from the group consisting of fluorine, chlorine, and bromine.

14. A process for converting ethylene glycol diacetate to vinyl acetate comprising:
admixing an ethylene glycol diacetate-containing feedstock with a substantially non-reactive compound having a normal boiling point less than that of ethylene glycol diacetate to provide a liquid-liquid mixture;
passing the liquid-liquid mixture through a hydrodynamic cavitation apparatus to provide cavitation features to provide a two-phase liquid-gas mixture containing the ethylene glycol diacetate in the liquid state and the non-reactive compound in the gaseous state in the form of vapor phase bubbles by reducing the pressure to at or below the liquid/vapor pressure threshold for the non-reactive compound then collapsing the bubbles resulting in localized areas of increased pressure and temperature sufficient to convert at least a portion of the ethylene glycol diacetate to vinyl acetate and acetic acid and provide a product mixture; and
separating vinyl acetate from the product mixture.

15. The process of claim 14 wherein the cavitation apparatus is a stationary or rotational cavitation apparatus.

16. The process of claim 15 wherein the cavitation apparatus is an orifice or vortex cavitation apparatus.

17. The process of claim 14 wherein the cavitation apparatus has two or more cavitation zones in series.

18. The process of claim 14 wherein the non-reactive compound comprises a hydrocarbon having from 5 to 16 carbons and a normal boiling point between about 80° and 220° C.

19. The process of claim 14 wherein the non-reactive compound is present in an amount of about 0.5 to 10 mass percent of the ethylene glycol diacetate-containing feedstock.

20. The process of claim 14 wherein the temperature of the liquid-liquid mixture as it is passed to the cavitation apparatus is between about 50° and 150° C.; and
wherein the pressure of the liquid-liquid mixture as it is passed to the cavitation apparatus is from about 200 to 50,000 kPa absolute.
